(19) United States
(12) Patent Application Publication
(10) Pub. No.: US 2025/0261908 A1
(43) Pub. Date: Aug. 21, 2025

(54) ANYTIME VITALS ANYWHERE AVA

(71) Applicant: Nishant NARAYANAN, Palatine, IL (US)

(72) Inventor: Nishant NARAYANAN, Palatine, IL (US)

(21) Appl. No.: 18/582,439

(22) Filed: Feb. 20, 2024

(51) Int. Cl. A61B 5/00

(52) U.S. Cl. CPC A61B 5/7264; A61B 5/6824; A61B 5/6826; A61B 5/743; A61B 5/746

(57) ABSTRACT

Apparatus and methods are disclosed for monitoring, tracking, and storing vital signs, including adjustable wristband and ring to fit the patient and/or user and a sensor, motherboard, and web platform and/or mobile dashboard to notify the patient/user, health care team, and family members when the patient/user's vital sign is out of range, and display and store vital signs data for patient/user, health care team, and family members to access during medical care or daily life.

### TECHNICAL FIELD

The present disclosure relates to body vital sign monitors, and more particularly to apparatuses and methods of monitoring vital signs, storing vital sign data, and notifying users when vital signs are out of range.

### BACKGROUND

Accurate and consistent vital sign measurements are critical to patient care, particularly for the patient's health care team and family. Although monitoring devices exist for body temperature and other vital signs, a need exists for a holistic health care monitoring and notification device that can track a wide range of vital signs in real time, remotely share data with clinicians, family members and other authorized third parties, store the vital signs data, and notify users when vital signs are out of range.

### SUMMARY

In one aspect, the invention provides an apparatus that syncs with a web-based or mobile platform to display and store a user's vital signs, including heart rate, body temperature, oxygen level, mental state resilience, and hydration levels. In some embodiments, the apparatus includes a sensor attached to a ring that is configured to measure various vital signs, a motherboard attached to a wristband, and a cable connecting the sensor to the motherboard.

In some embodiments, the apparatus connects to a mobile dashboard or web platform. The mobile dashboard or web platform contains an input screen for the user to input unique, customizable minimum and maximum values for their vital signs. The mobile dashboard or web platform also contains input screens to set up notifications and grant access to authorized third parties, such as the user's health care team and family. The mobile dashboard or web platform also contains an input screen to customize the unique notification, alert, or warning message related to the vital sign. The mobile dashboard or web platform will automatically send the unique notification, alert, or warning when the user's vital sign is outside the designated range.

In certain aspects, the present disclosure provides a device and non-transitory computer readable medium storing program instructions that are executable to monitor, track, and store vital signs, comprising: an input screen on a mobile dashboard or web platform to enter a minimum value and a maximum values for vital signs; a vitals sensor that reads vital sign data at regular intervals; a ring in which the vitals sensor is placed; a connective cable that sends the vital sign data from the sensor; a motherboard that receives the vital sign data from the sensor through the connective cable; and a wristband in which the motherboard is placed; wherein the motherboard contains a coding algorithm that calculates and compares the vital sign data to the inputted minimum value and the inputted maximum values for vital signs; wherein the motherboard sends the vital sign data to the mobile dashboard or web platform through a communication network; wherein the mobile dashboard or web platform processes and displays the vital sign data; and wherein the mobile dashboard or web platform displays an alert when the vital sign data is outside of the inputted minimum value and the inputted maximum values for the vital signs. In certain embodiments, the device and non-transitory computer readable medium stores additional program instructions that are executable to display historical trends of vitals data in a chart, graph, or timeline. In certain embodiments, the device and non-transitory computer readable medium stores additional program instructions that are executable to determine when vital signs have been increasing or decreasing over time; wherein the consistent variability in vital sign over time indicates the severity of the associated health complication; and send an alert or notification immediately, in two minutes, or in thirty minutes depending on the severity of the associated health complication.

In certain aspects, the present disclosure provides a method of monitoring, tracking, and storing vitals data, comprising: reading vitals data with a vitals sensor at regular intervals; sending vitals data from the vitals sensor to a motherboard through a connective cable; calculating and comparing vitals data through a coding algorithm within the motherboard; sending the vitals data to a mobile dashboard or web platform through a communication network; and displaying the vitals data on the mobile dashboard or web platform. In certain embodiments, the method comprises inputting a minimum value and a maximum value associated with the vital sign; and sending an alert or notification when the vitals data is out of the range determined by the input minimum value and the input maximum value.

### BRIEF DESCRIPTION OF THE DRAWINGS

The following figures depict illustrative embodiments of the invention.

FIG. 1 is an illustration showing a configuration of an exemplary apparatus as worn by a user, according to some embodiments of the present disclosure.

FIG. 2 is an illustrative diagram showing an exemplary apparatus according to some embodiments of the present disclosure.

FIG. 3 is an illustrative flowchart showing the process of using the apparatus and mobile dashboard or web platform, according to some embodiments of the present disclosure.

FIG. 4 is an exemplary illustration of the input screen for the user to input customizable minimum and maximum values for monitored vital signs.

FIG. 5 is an exemplary illustration of the mobile dashboard platform.

FIG. 6 is an exemplary illustration of mobile notifications when vital signs are outside of the user designated minimum and maximum range of the monitored vital signs.

FIG. 7 is an exemplary illustration of the mobile dashboard notification enablement screen.

FIG. 8 is an exemplary illustration of the mobile dashboard screen for the user or other qualified representative to grant access to others to view the user's vitals information and stored data.

FIG. 9 is an exemplary illustration of the mobile dashboard screen to customize warning, alert, or notification messages when vitals are outside of the user's designated minimum and maximum vitals range.

FIG. 10 is an exemplary illustration of an email alert when the user's vital sign is out of the user's designated range.

FIGS. 11A-B are exemplary illustrations of a ring extension configuration of the apparatus.

FIGS. 12A-B are exemplary illustrations of a bracelet configuration of the apparatus.

### DETAILED DESCRIPTION

The ability to accurately measure and track human body vital signs is critical to determining, evaluating, and improving a person's health and wellness. Additionally, accurate vital measurements are important to health care providers when determining a patient's medical needs. The use of vital sign monitors and trackers have proven to be a great tool for consumers and health care providers. Currently, there is not a device that can simultaneously track a user/patient's vital signs and share vital signs data with the user/patient, family members, and health care team. For example, existing vital sign monitors are limited to tracking body temperature only as a fever spikes or are limited to use for infants. Additionally, existing monitors do not have features capable of remote diagnosis or continuous monitoring.

The present disclosure solves these problems and is drawn to apparatuses, devices, systems, and methods of tracking a user's vital signs, displaying and storing the user's vital signs on a mobile dashboard or web platform, and notifying the user when vital signs are out of range. In some embodiments, the mobile dashboard or web platform allows the user to input minimum and maximum values for each vital sign and can send warnings, notifications, or alerts if the user's vital signs are outside of the inputted minimum and maximum values. In some embodiments, the mobile dashboard or web platform stores historical vital signs data that the user and other authorized third parties can access and creates seamless communication between the patient/user, the health care team, and family members. In some embodiments, the present disclosure is directed to providing a medical device that a healthcare team, including physicians, nurses, and home health aides, can use to monitor and provide care to a patient.

In some embodiments, the apparatus of the present invention can monitor various vital signs, such as body temperature, pulse rate, oxygen levels, mental state resilience, hydration, and/or some combination of these vitals. Mental state resilience is determined based on heart-rate variability. For example, if the patient's heart rate is frequently changing, the patient will demonstrate low mental state resilience. Hydration is determined based on oxygen levels. For example, low oxygen levels correlate to low hydration.

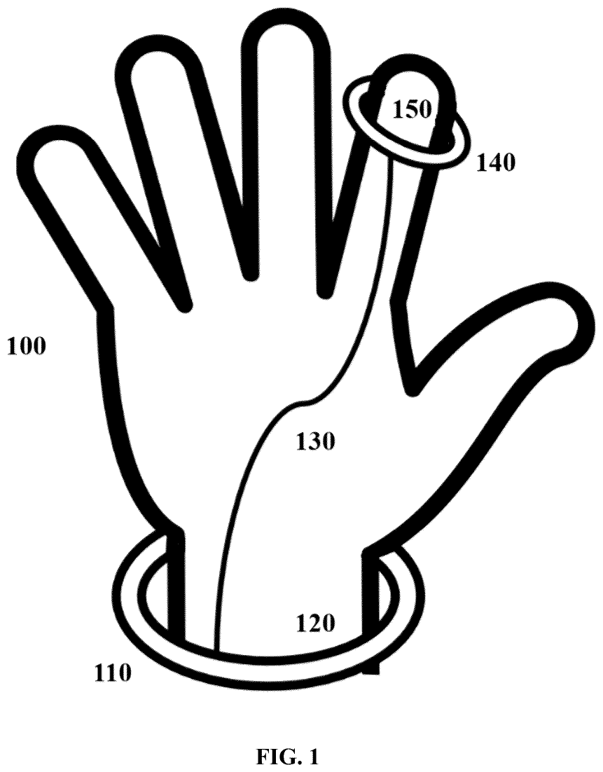

The present disclosure describes in one embodiment an apparatus and mobile dashboard or web platform and methods of use of the apparatus with the web-based application to monitor the body temperature, heart rate, average oxygen level, and body movement of a human subject. FIG. 1 is an illustration showing a configuration of an exemplary apparatus 100 as worn by a user, according to some embodiments of the present disclosure. The apparatus 100 can include a vitals sensor 150, a ring 140, a cable 130, a motherboard 120, and a wristband 110. Vitals sensor 150 can include mechanical components and/or electronic sensors that respond to body vital signs, such as body temperature, oxygen level, heart rate, and body movement. Ring 140 can be made of an adjustable material to fit the user's finger. Motherboard 120 can include mechanical and/or electrical components that store data. Vitals sensor 150 is placed within the ring 140. For most accurate vital sign measurements, the vitals sensor 140 should be placed near the pad of the index finger. Motherboard 120 can include a battery or connection port to a power source. Cable 130 can be made of a material that transfers power from motherboard 120 to vitals sensor 150. Exemplary materials include, jumper wires, aluminum electrical wires, copper electrical wires or other conductive connective wires or cables. Wristband 110 can be made of a material capable of storing motherboard 120 and connecting to jumper 130. Exemplary materials include, silicone, rubber, textile fabrics or other non-conductive materials.

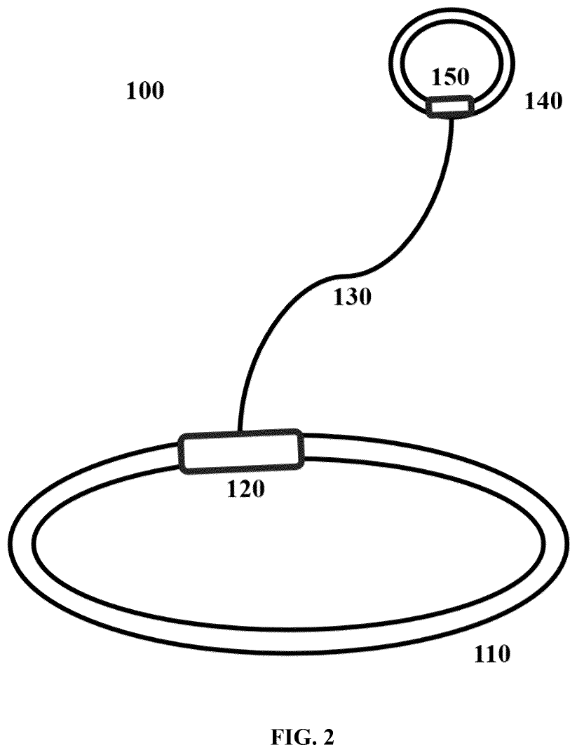

FIG. 2 is an illustration showing a configuration of an exemplary apparatus 100. Vitals sensor 150 can monitor the vital signs of the user. Vitals sensor 150 collects the vitals data and sends the data to motherboard 120. Motherboard 120 sends the data to the web-based application and provides power to the sensor by sending electrical supply to the sensor. In some embodiments, motherboard 120 receives power from a computer. In some embodiments, motherboard 120 receives power from a portable power-bank. In some embodiments, motherboard 120 receives power from a battery. In some embodiments, the ring 140 may be of various sizes and materials (e.g., silicone, rubber, textile fabric, or other non-conductive material). The size of ring 140 can be adjusted to fit the size of the user's finger. In some embodiments, the wristband 110 may be of various sizes and materials (e.g., silicone, rubber, textile fabric, or other non-conductive material). In some embodiments, the jumper 130 may be of various lengths and materials. The length of jumper 130 can be adjustable based on the size of the user's hand. The jumper must be connected to the input and output ports of the motherboard and sensor in order for the motherboard to power the sensor.

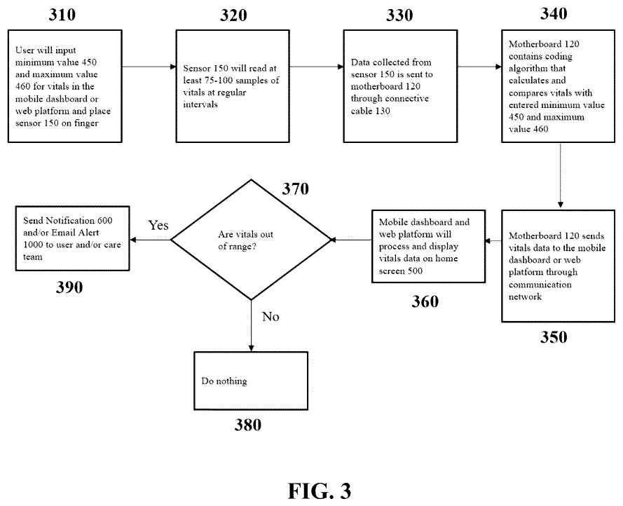

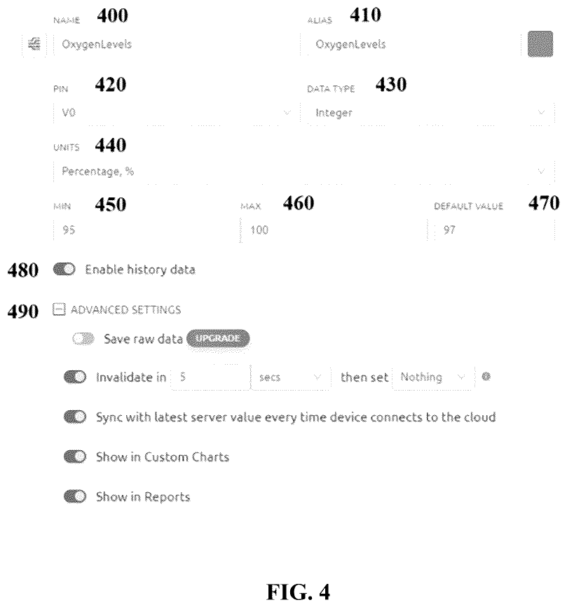

FIG. 3 is an illustrative flowchart showing the process of using the apparatus and w mobile dashboard or web platform. The apparatus can send vitals information to either/both a mobile dashboard or web platform. First, as shown in box 310, the user or authorized third party, such as a healthcare provider, will input customizable minimum vital value 450 and maximum vitals value 460 in the mobile dashboard or web platform as shown in FIG. 4 and place vitals sensor 150 on the user's finger. Next, as shown in box 320, the vitals sensor will read, for example, at least 75 to 100 samples of vitals information at regular intervals. In other examples, the vitals sensor reads 25 to 50 samples, 50 to 75 samples, or over 100 samples. In some embodiments, the vitals sensor will collect vitals data at regular intervals (e.g., 30 second, 1 minute, 90 second, and 2 minute intervals). Then, as shown in box 330, the vitals data collected from sensor 150 is sent to motherboard 120 through the connective cable 130. In some embodiments, each sample is sent sensor 150 to motherboard 120 individually. Motherboard 120 a coding algorithm that averages the collected samples (e.g., 75 to 100 samples). The coding algorithm within motherboard 120 then compares the average of the collected vitals data with entered minimum value 450 and maximum value 460, as shown in box 340. Then, as shown in box 350, motherboard 120 sends vitals data to the mobile dashboard or web platform through a communication network such as, for example, cable networks, public networks (e.g., the Internet), wireless networks, cellular networks, metropolitan area networks (MANs), wide area networks (WANs), local area networks (LANs), personal area networks (PANs), Bluetooth connection, and Wi-Fi connections. Then, as shown in box 360, the mobile dashboard or web platform will process and display the vitals data on home screen 500. The mobile dashboard or web platform will analyze whether the vitals are out of range, as shown in box 370. The mobile dashboard or web platform will do nothing when vitals are not out of range, as shown in box 380. Otherwise, as shown in box 390, the mobile dashboard or web platform will send notification 600 or email alert 1000 to the user and/or care team when vitals are out of range.

In some embodiments, the user can manually track and monitor their vitals. The patient or user can use the mobile dashboard or web platform to refresh their vitals data on-demand. The apparatus monitors the vitals multiple times over a period of time (e.g., in 30 second, 1 minute, 90 second, or 2 minute intervals) and the mobile dashboard or web platform will display the most recently monitored data on home screen 500.

FIG. 4 is an illustration depicting the input screen for the user of the apparatus, or an authorized third party such as a healthcare provider, to input customized, personal vital sign information. The input screen displays the pre-populated name of the vital sign 400 and alias of the vital sign 410. The input screen also displays the pre-populated unique identifier 420 assigned to the vital sign (e.g., PIN value). In some embodiments, the unique identifier 420 is a particular symbol used to identify the individual vital sign that the system determines and cannot be customized by the user. The input screen also displays the data type 430 (e.g., integer). In some embodiments, the data type 430 is determined by the system, cannot be customized by the user, and identifies the type of data the system is collecting. The input screen also displays the units 440 (e.g., percentage, %). In some embodiments, the units 440 is determined by the system, cannot be customized by the user, and identifies the type of unit of data type 430 collected by the system. In some embodiments, the input screen will have an option to input a minimum value 450 and maximum value 460 for the vital sign. In some embodiments, the input screen will also have a pre-populated default value 470 based on average values of the vital sign. In some embodiments, the input screen will have a toggle for the user to enable history data 480 and advanced settings 490. In some embodiments, enabling history data 480 allows the mobile dashboard and/or web platform to store historical vitals data. An example of advanced settings 490 includes displaying “Nothing” or “No Data” when the mobile dashboard or web platform has not received data in a set amount of time (e.g., 1 minute). Another example of advanced settings 490 includes syncing with latest server value every time device connects to the cloud. When this setting is turned on, a device that has been offline will retrieve the latest value collected from the sensor 150 when the system turns back online. The motherboard 120 will then collect data regularly as shown in FIG. 3. Another example of advanced settings 490 includes showing vitals data in custom charts and reports for the user, health care team and/or doctor to see vitals data over a period of time. In some embodiments, the collected vitals data from box 330 can be plotted in a graph to show the patient's progress related to that particular vital sign. In some embodiments, the collected vitals data from box 330 can be used to show improvement or decline related to the particular vital sign, as well as the rate of acceleration or deceleration of the improvement or decline. In some embodiments, the collected vital signs from box 330 can be used to provide an overview of a patient's vital signs mapped against the patient's circadian rhythm. Over time, if the vital signs are not following the patient's circadian trend, the system can alert the health care team of possible sleep disturbance.

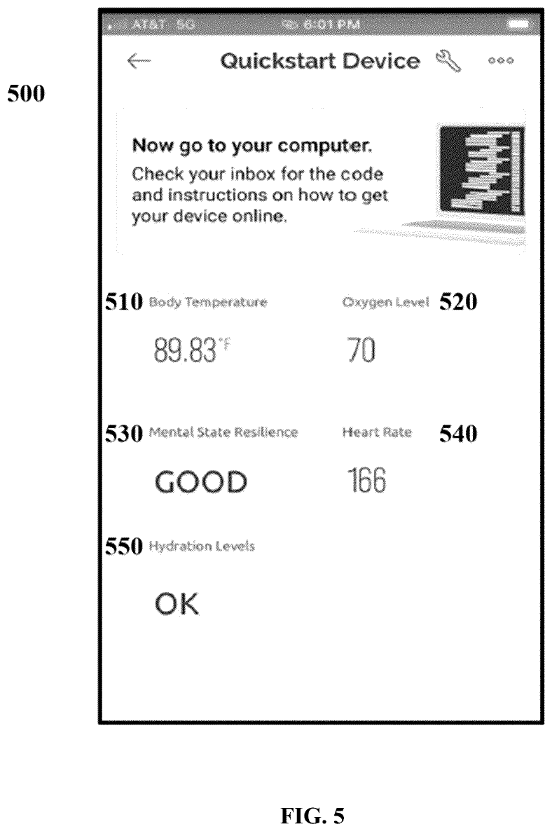

FIG. 5 is an illustration depicting an exemplary home-screen for the mobile dashboard. In some embodiments, the home screen 500 will display body temperature 510, oxygen level 520, heart rate 530, mental state resilience 540, and hydration levels 550. The data shown on this screen can be the last sample collected by vitals sensor 150. In other embodiments, it can display the average value of collected samples over the previous minute, hour, 6-hour period, 12-hour period, or day. A display reading of “GOOD” for mental state resilience indicates minimal or no variability in heart rate. A display reading of “POOR” for mental state resilience indicates abnormal variability in heart rate. A display reading of “OK” for hydration levels indicates regular body temperature and regular oxygen levels. A display reading of “POOR” for hydration levels indicates high body temperature and low oxygen levels.

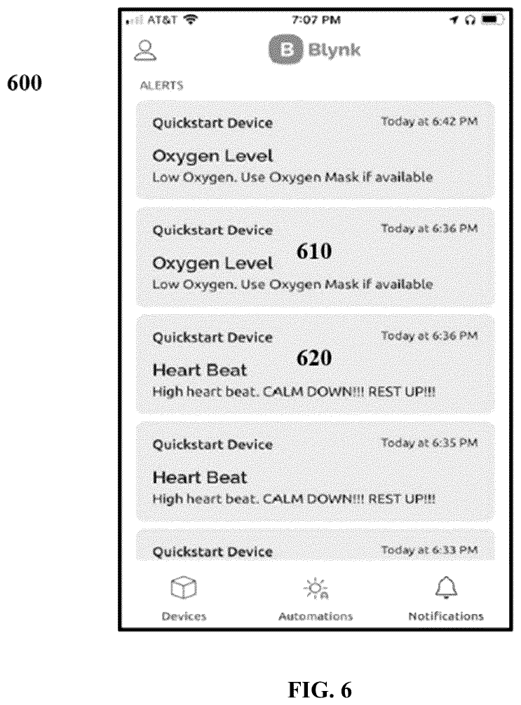

FIG. 6 is an illustration depicting an exemplary display of mobile device notifications 600 in the mobile dashboard. Notifications are sent to the mobile dashboard when vital signs are outside of the inputted range. Exemplary notification for low oxygen level 610 displays an alert, such as “Oxygen Level: Low Oxygen. Use Oxygen Mask if available.” Exemplary notification for high heart rate 620 displays an alert, such as “Heart Beat: High heart beat. Calm down! Rest up!” In some embodiments, a notification is sent when oxygen levels drop for over 30 seconds, one minute, two minutes, five minutes, ten minutes, or longer. In some embodiments, a notification is sent when hydration levels decrease over the course of 10 minutes, 20 minutes, 30 minutes, 40 minutes, 50 minutes, 60 minutes, or longer. In some embodiments, a notification is sent immediately when heart rate variability indicates atrial fibrillation or other severe cardiac conduction abnormality.

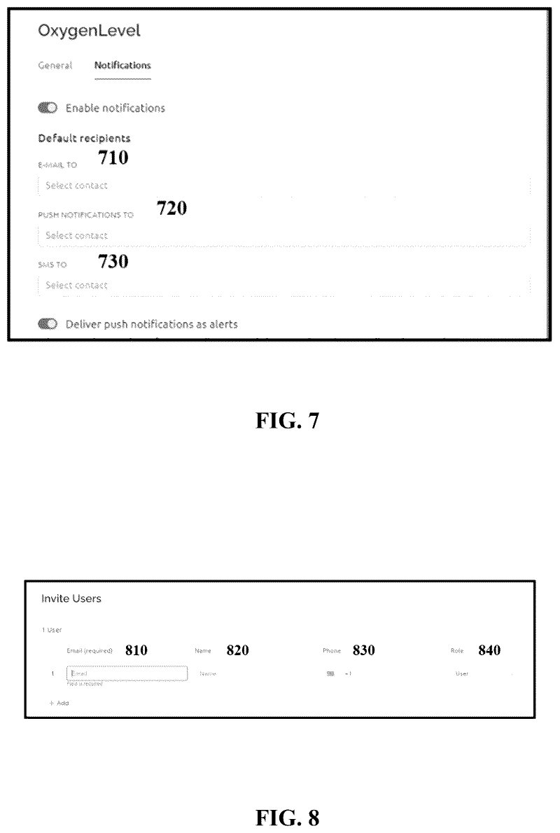

FIG. 7 is an illustration depicting an exemplary input screen for the user or authorized third party to set up notification alerts 600 when the user's vital signs are out of the minimum value 450 and maximum value 460 range for a designated period of time the user or authorized third party inputted in the display of FIG. 4. In some embodiments, the input screen for setting up notifications includes a text box to input email contact information 710 for the user and authorized third parties to receive notifications. In some embodiments, the input screen for setting up notifications includes a text box to input mobile application push notifications 720 for the user and authorized third parties to receive notifications. In some embodiments, the input screen for setting up notifications includes a text box to input SMS text contact information 730 for the user and authorized third parties to receive notifications.

FIG. 8 is an illustration depicting an exemplary input screen for the user or authorized third party to grant access to the user's vital sign information to other individuals. In some embodiments, the input screen includes a text box for the authorized third party's email address 810, the authorized third party's name 820, the authorized third party's phone number 830, and the role or relation of the authorized third party to the user 840. In some embodiments, the role of the user is “admin” and the role of other authorized third party's is “user.”

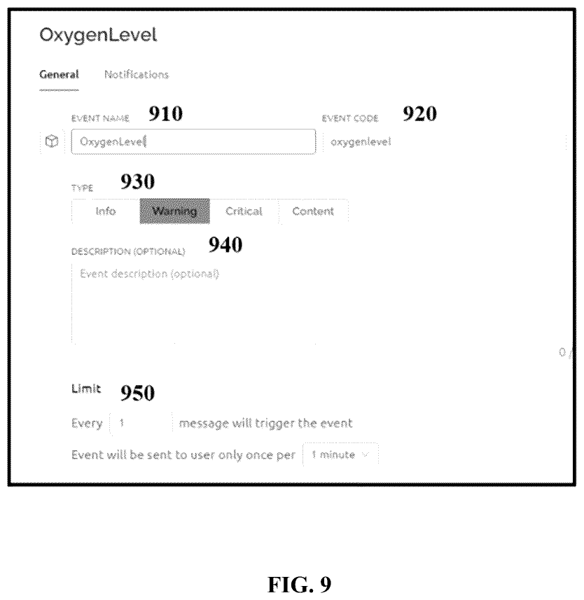

FIG. 9 is an illustration depicting an exemplary input screen for the user to customize the warning, alert, and notification messages that the user and authorized third parties will receive. In some embodiments, the name of the vital sign 910 and the event code 920 is pre-populated. In some embodiments, the type of alert 930 can be selected from options including “info,” “warning,” “critical,” and “content.” In some embodiments, the input screen for customizing warning and notification messages will include a text box for a custom description 940. For example, this is where the message shown in FIG. 66, “CALM DOWN!!! REST UP!!!” can be included. In some embodiments, a text box is available for the user to input the limit 950 of messages triggered to avoid excessive notifications if the associated vital sign is out-of-range for a long period of time. For example, if the user's oxygen level is low for a long period of time, the system's default setting is to send an alert or notification for each collection of samples sent from the sensor 150 to motherboard 120. The limit 950 will reduce the number of alerts or notifications sent to the designated amount (e.g., 1 notification per minute) even if there are multiple readings that are out of range within that designated time period.

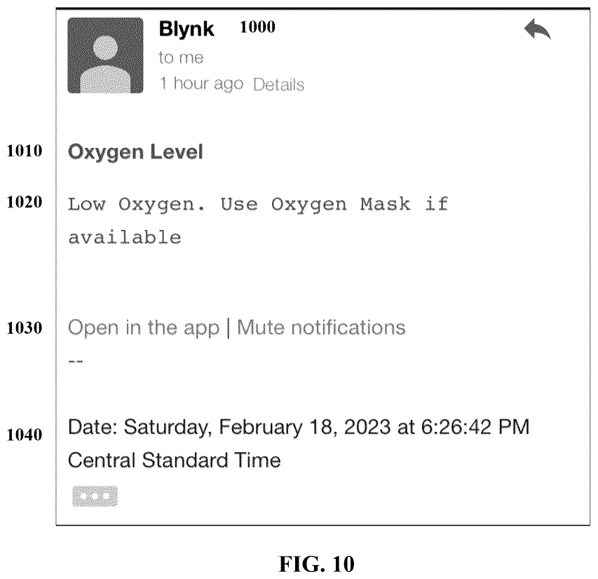

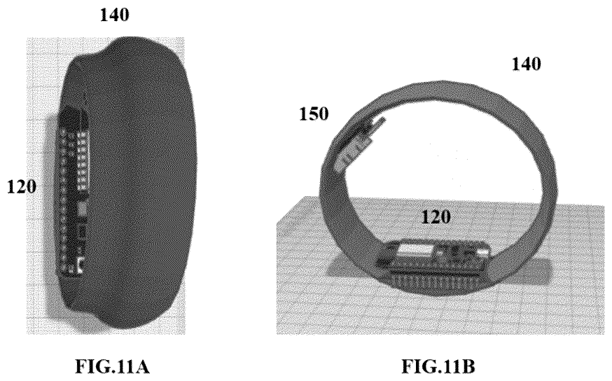

FIG. 10 is an illustration depicting an exemplary email alert the user or authorized third party will receive. In some embodiments, the name of the vital sign 1010, the custom alert or message 1020, the option to open the notification in the app or mute notifications 1030, and the date and time of the notification 1040 will appear in the email. The email alert will depict the customized message the user entered in the input screen of FIG. 9.

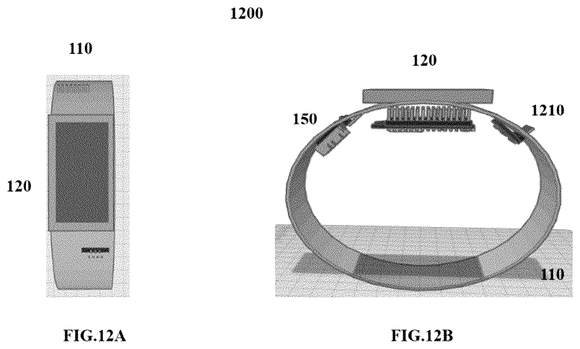

FIG. 11 is an illustration depicting a ring extension 1100 of the apparatus. Ring extension 1100 is a different configuration of apparatus 100. Ring extension 1100 only includes ring 140, which holds sensor 120 and motherboard 150. In some embodiments, ring extension 1100 includes a hidden connective cable 130 within the material of ring 140. In some embodiments, sensor 120 sends data to motherboard 150 through the material of ring 140. FIG. 11A depicts an inside view of the ring extension, where motherboard 120 is visible. FIG. 11B is a side view of the ring extension, where motherboard 120 and vitals sensor 150 are visible on the inside of the ring.

FIG. 12 is an illustration depicting a bracelet extension 1200 of the apparatus. Bracelet extension 1200 is a different configuration of apparatus 100. Bracelet extension 1200 only includes wristband 110, which holds sensor 120 and motherboard 150. In some embodiments, bracelet extension 1200 includes a hidden connective cable 130 within the material of the wristband 110. In some embodiments, sensor 120 sends data to motherboard 150 through the material of wristband 110. FIG. 12A is an outside view of the bracelet where the motherboard 120 is visible. FIG. 12B is a side view of the bracelet wearable, where motherboard 120, vitals sensor 150, and bracelet adjustment clip 1210 is visible.

Patients can use any one of devices 100, 1100 or 1200 to monitor their vitals on-demand. Significant or critical changes to vitals can occur in between active vitals readings. Using the present invention, a patient can monitor their own vitals at any time and from any location, as long as the patient is wearing the device. If the patient's vitals are out of the designated normal range for a designated period of time, the application will send an alert to the patient. This allows the patient to quickly respond and take the necessary actions to return to their normal range of vitals. Based on the unique minimum and maximum vital sign values, the patient has access to health indicators like the mental state resilience and the hydration levels provided by the application. This will prevent patients from suffering from catastrophic illnesses, such as heart attacks, as the invention will be able to detect early symptoms and warn the patient.

If given access by the patient, doctors can use the invention to monitor their patients' vital signs. If granted access by the patient to the patient's vital sign information, doctors can give more accurate diagnoses and treatment plans. Doctors can also provide patients with well-informed recommendations based on the patient's vital readings. The invention will also improve concierge medicine because it will help build stronger connections between the patient and doctor. Because the doctor can view the patient's real-time vital sign information at any time from any location, the doctor will be able to provide more personalized care to the patient. The mobile application captures the historical data and enables doctors to view historical trends, which can increase efficiency and the accuracy of the diagnosis and treatment in telehealth and home health. With access to historical patient vital data, doctors in the concierge medicine industry will have immediate information related to the patient, improving the efficiency and effectiveness of concierge medicine. Additionally, the patient can directly share the vitals data with their doctor. If any monitored vitals are out of range, a notification will also be sent to the doctor so they can offer any treatment or care if necessary.

Nurses typically check a patients' vitals before the doctor checks on the patient. However, when nurses enter the treating room, patients may be carrying a contagious infection or disease. Nurses are at a high risk of infection due to the close proximity in which they care for the patient. The present invention can help solve this issue as the nurse can use the product to remotely and accurately monitor the vitals in real time. They can also view a patient's historical data, which gives the nurse important information about the patient and provides the nurse with information to share with doctors, if necessary. Authorized nurses will also receive a notification if one of the vitals are out of range so that corrective actions can be taken. With this invention, nurses will be able to care for all their patients without risking their own health.

When individuals contract diseases, their family members and loved ones are often the people who care for them. For children especially, family members and other loved ones need to come into close contact with sick family members to monitor their vital measurements. Because common diseases are contagious, this puts the caretaker at a high risk of contracting the disease. The present invention can be used so that family members and caretakers preserve their own health and can simultaneously monitor the family members' vitals in real-time. Additionally, the application provides notifications when the patient's vitals are out-of-range, which makes providing care for the patient easier and more efficient for the caretaker.

The present invention is a unique solution that is targeted towards the amalgamation of the care team (i.e., doctors, nurses, care team) by providing seamless communications and accurate vital sign information. The care team will highly benefit from because they can each monitor vitals from a remote location without risking exposure to the sick patient. The patient has the sole discretion to share their information with who they choose to have access to the vitals. Additionally, the web-based application displays all the historical data in real time for the care team to use.

1. A device and non-transitory computer readable medium storing program instructions that are executable to monitor, track, and store vital signs, comprising:
an input screen on a mobile dashboard or web platform to enter a minimum value and a maximum values for vital signs;
a vitals sensor that reads vital sign data at regular intervals;
a ring in which the vitals sensor is placed;
a connective cable that sends the vital sign data from the sensor;
a motherboard that receives the vital sign data from the sensor through the connective cable;
and a wristband in which the motherboard is placed;
wherein the motherboard contains a coding algorithm that calculates and compares the vital sign data to the inputted minimum value and the inputted maximum values for vital signs;
wherein the motherboard sends the vital sign data to the mobile dashboard or web platform through a communication network;
wherein the mobile dashboard or web platform processes and displays the vital sign data;
and wherein the mobile dashboard or web platform displays an alert when the vital sign data is outside of the inputted minimum value and the inputted maximum values for the vital signs.

2. A device and non-transitory computer readable medium of claim 1, storing additional program instructions that are executable to:
display historical trends of vitals data in a chart, graph, or timeline.

3. A device and non-transitory computer readable medium of claim 1, storing additional program instructions that are executable to:
determine when vital signs have been increasing or decreasing over time;
wherein the consistent variability in vital sign over time indicates the severity of the associated health complication;
and send an alert or notification immediately, in two minutes, or in thirty minutes depending on the severity of the associated health complication.

4. A method of monitoring, tracking, and storing vitals data, comprising:
reading vitals data with a vitals sensor at regular intervals;
sending vitals data from the vitals sensor to a motherboard through a connective cable;
calculating and comparing vitals data through a coding algorithm within the motherboard;
sending the vitals data to a mobile dashboard or web platform through a communication network;
and displaying the vitals data on the mobile dashboard or web platform.

5. A method of claim 1, comprising:
inputting a minimum value and a maximum value associated with the vital sign;
and sending an alert or notification when the vitals data is out of the range determined by the input minimum value and the input maximum value.
